(19) United States
(12) Patent Application Publication
(10) Pub. No.: US 2021/0357846 A1
(43) Pub. Date: Nov. 18, 2021

(54) METHOD FOR MONITORING AN AUTOMATION SYSTEM

(71) Applicant: Endress+Hauser Process Solutions AG, Reinach (CH)

(72) Inventors: Werner Thoren, Steinen (DE); Robert Kölblin, Lörrach (DE); Christian Isler, Laufen (CH); Hans-Jürgen Huber, Freiburg (DE)

(21) Appl. No.: 16/768,630

(22) PCT Filed: Oct. 2, 2018
(86) PCT No.: PCT/EP2018/076842

§ 371 Date: May 29, 2020

(30) Foreign Application Priority Data
Nov. 30, 2017 (DE) 10 2017 128 437.0

(51) Int. Cl. G06Q 10/06; G05B 19/042; H04W 4/029

(52) U.S. Cl. CPC G06Q 10/063112; G06Q 10/06313; G05B 2219/25428; H04W 4/029; G05B 2219/23406; G05B 19/0428

(57) ABSTRACT

The invention resides in a method for maintaining a measuring location (ML1, ML2, ML3) in an automated plant (A), wherein capability information (CI) of persons (P1, P2 P3) are registered and stored in a service platform (SP), wherein the capability information (CI) includes information concerning objects brought along by the person (P1, P2, P3), health of the person (P1, P2, P3) and/or safety equipment worn by the person (P1, P2, P3), comprising:
noting a case of service or maintenance of the measuring location (ML1, ML2, ML3);
creating a service or maintenance order (SO), which includes objects required for caring for the case of service or maintenance, health requirements and/or requirements regarding safety equipment;
calculating a degree of suitability of persons (P1, P2, P3) stored in the service platform (SP) upon noting a case of service or maintenance for the measuring location (ML1, ML2, ML3), wherein the degree of suitability provides a measure of agreement of capability information (CI) for persons (P1, P2, P3) stored in the database with the required objects, the requirements for health and/or the requirements as regards safety equipment contained in the service or maintenance plan; and
delivering the service or maintenance order (SO) to at least one of the persons (P1, P2, P3) stored in the service platform (SP), who has for the noted case of service or diagnosis a degree of suitability, which is equal to or greater than a predetermined degree of suitability.

The invention relates to a method for maintaining a measuring location in an automation system, especially in an automated plant.

Known from the state of the art are field devices, which are used in industrial plants. Field devices are applied in automation technology, particularly in manufacturing automation. Referred to as field devices are, in principle, all devices, which are applied near to a process and which deliver, or process, process relevant information. Thus, field devices are used for registering and/or influencing process variables. Serving for registering process variables are sensor systems. Such are used, for example, for pressure- and temperature measurement, conductivity measurement, flow measurement, pH measurement, fill level measurement, etc. and register the corresponding process variables, pressure, temperature, conductivity, pH value, fill level, flow, etc. Used for influencing process variables are actuator systems. Such are, for example, pumps or valves, which can influence the flow of a liquid in a tube or the fill level in a container. Besides the above mentioned measuring devices and actuators, referred to as field devices are also remote I/Os, radio adapters, and, in general, devices, which are arranged at the field level.

A large number of such field devices are produced and sold by the Endress+Hauser group of companies.

In modern industrial plants, field devices are, as a rule, connected with superordinated units via communication networks, such as, for example, fieldbusses (Profibus®, Foundation® Fieldbus, HART®, etc.). The superordinated units are control units, such as, for example, a PLC (programmable logic controller). The superordinated units serve, among other things, for process control, as well as for start-up of the field devices. The measured values registered by the field devices, especially by their sensors, are transferred via the particular bus system to one or more superordinated unit(s), which, in given cases, further process the measured values and forward them to the control station of the plant. The control station serves for process visualizing, process monitoring and process control via the superordinated units. In addition, also a data transmission from the superordinated unit via the bus system to the field devices is required, especially for configuration and parametering of field devices as well as for operating actuators.

In the course of Industry 4.0 and IIoT (“Industrial Internet of Things”), data produced by field devices are frequently collected directly from the field with the help of so-called data conversion units, which are referred to, for example, as “edge devices” or “cloud gateways”, and transferred automatically to a central cloud-capable service platform, in which an application is located. This application, which, among other things, provides functions for visualizing and additional processing of the data stored in the service platform, can be accessed by a user by means of the Internet.

By means of these methods, monitoring of the electronic components of the plant—thus, the field devices and control units—is possible. In the case of malfunction, diagnostic messages are created by the affected field devices and sent to the control station of the plant, and/or to the service platform. Likewise, also diagnostic messages regarding the communication networks are created. Indirectly, also malfunctions of plant components, such as tank and pipelines, for example, in the case of accretion formation or leakage, can be determined. These malfunctions are determined, for example, via changed measuring characteristics of the field devices.

In the case of arrival of a diagnostic message at the control station of the plant, such is currently often still manually checked and a service or maintenance plan for the component in question created. This service or maintenance plan is then assigned to a service technician, who finds the component in question and performs the maintenance, or service. Depending on the type of component, the malfunction and the location of the component, the service technician must pay attention to bring along the right maintenance tools, servicing devices and/or safety equipment. For example, if difficult environmental conditions in the form of poisonous vapors exist at a measuring location due to the utilized process media, then the service technician must bring along suitable safety equipment. If a measuring location is located at a great height and, for example, is only reachable via a ladder, then, in given cases, not every service technician has the requisite health for reaching the measuring location.

In summary, it can be said that there currently lies between the occurrence of a failure and its removal sometimes a large time span, which can be further lengthened by possible multiple variables, for example, when not all tools required for removal of a failure are brought along by the service technician.

Based on the above, an object of the invention is to provide a method, which enables accelerating the removal of a failure in an automated plant.

The object is achieved by a method for maintaining a measuring location in an automated plant, wherein there exist at the measuring location at least one plant component, especially a container and/or a pipeline, in which a process medium is located at least at times, at least one field device, which is embodied for registering and/or influencing process variables of the process medium and at least one communication means, which is embodied for transferring to a control station of the plant the process variables registered by the field device and/or diagnostic information produced by the field device, comprising:
registering capability information of a person located in the plant or entering the plant, wherein the capability information includes information concerning objects brought along by the person, health of the person and/or safety equipment worn by the person;
sending to and storing the registered capability information of the person in a service platform, especially a cloud based service platform;
noting a case of service or maintenance of the measuring location;
creating a service or maintenance order, which includes objects required for caring for the case of service or maintenance, health requirements and/or requirements regarding safety equipment;
calculating a degree of suitability of persons stored in the service platform upon noting a case of service or maintenance for the measuring location, wherein the degree of suitability provides a measure of agreement of capability information for persons stored in the database with the required objects, the requirements for health and/or the requirements as regards safety equipment contained in the service or maintenance plan; and
delivering the service or maintenance order to at least one of the persons stored in the service platform, who has for the noted case of service or diagnosis a degree of suitability, which is equal to or greater than a predetermined degree of suitability.

A great advantage of the method of the invention is that with its help a case of malfunction at a measuring location can be rapidly removed. Referred to as a case of malfunction at a measuring location is a case of malfunction at at least one component of the measuring location, for example, at one of the field devices, at one of the plant components, for example, at a container, a pipeline, a valve, etc., or at the communication means. In this way, upon occurrence of the case of service or maintenance, a service or maintenance order is created, which is delivered to a suitable person in the plant, for example, a service technician. Such a person carries especially a service unit or a mobile end device, to which the service or maintenance plan is delivered. The service unit, or the mobile end device, as the case may be, is, in such case, connected with the service platform by means of the Internet.

The terminology, communication means, refers to a communication network. Such can be embodied wired. In such case, the communication means is, for example, a fieldbus of automation technology, for example, a PROFIBUS, Foundation Fieldbus, HART, etc. fieldbus, an Ethernet fieldbus, for example, PROFINET, or an IT communication network. Alternatively, the communication means is embodied wirelessly. In such case, the communication means is a wireless network, which uses as protocol, for example, a Wi-Fi, Bluetooth (LE), Zigbee, etc. protocol or a communication network, which uses a mobile radio network, for example, a GPRS, EDGE, LTE, 4G, 5G, etc. network.

The terminology “maintenance” refers to a method, in the case of which no particular case of malfunction has happened to a plant component, a field device or a communication means. It can, however, be provided that the plant components are routinely tested. In such case, there is, for example, stored in the service platform a table, which provides the points in time, when such routine testing should occur. Also software updates can be downloaded or newly designed replacement parts installed in the plant components.

A “case of service” refers to a method, in the case of which a concrete case of malfunction has happened to a plant component, a field device or a communication means.

The suitability a certain person for eliminating an arisen case of malfunction is ascertained via a degree of suitability. For this, first, all objects, which the person brings along, as well as the protective equipment of the person are inventoried. The objects brought along include especially tools, such as screwdrivers, pliers, keys, etc., however, also replacement parts for specific plant components, communication means and/or field devices, and electronic servicing devices, such as, for example, a laptop or an industrial tablet. The safety equipment includes especially protective jacket, protective helmet, protective glasses, insulated boots, etc. The degree of suitability includes, additionally, the health of the persons located in the plant. Furthermore, the degree of suitability includes also the professional competence of a person, for example, the education of the person.

For the calculating of the degree of suitability, which is especially performed electronically in the service platform, the objects required for removing the failure and the required safety equipment are ascertained. Moreover, it is ascertained whether the persons are constitutionally suitable to perform the maintenance, or service. Such is, for example, important, when the measuring location is difficult to reach, for example, when it is located at a great height. For calculating of the degree of suitability, for example, a relevant history is accessed. For example, similar cases of failure have been experienced, for which certain objects were required. The history can cover measuring locations, or even entire plants. Also, it is ascertained, whether the measuring location is located in a certain protection region (for example, an Ex region) of the plant, in which, for example, due to certain conditions at the location, such as, for example, explosive/aggressive gases, particular requirements for safety equipment of a service technician result.

Examples of field devices have already been given, by way of example, in the introductory part of the description.

In a preferred further development of the method of the invention, it is provided that after the noting of the case of service or maintenance, the current locations of those persons, who have a predetermined degree of suitability for the noted case of service or diagnosis, are registered and wherein the service or maintenance order is delivered only to those persons, who are located within a predetermined distance from the measuring location. In this way, it is assured that a person reaches an affected measuring location as rapidly as possible. Moreover, advantageously, persons, who are located further removed than the defined distance from the measuring location and, consequently, require a long time to reach the measuring location, receive no contact in the form of a delivering of the service or maintenance order and, thus, are not diverted from their current work.

For the case, in which none of the persons located within the defined distance has the predetermined degree of suitability, the defined distance is successively increased, until a person can be found, which has the predetermined degree of suitability.

An advantageous embodiment of the method of the invention provides that the locations are registered by means of a service unit or a mobile end device carried by the person. It can, in such case, be provided that the current locational position is ascertained in regular intervals recurringly and automatically from the service units, or the mobile end devices, and delivered to the service platform.

In a preferred further development of the method of the invention, it is provided that, upon successful removal of the case of service or maintenance, a certification thereof is delivered to the database by the person, who did the work. The case of service or maintenance can then be marked as “finished” in the service platform.

An advantageous embodiment of the method of the invention provides that in the case of no certification after a defined time interval after delivery of the service or maintenance order, a repetition of the method steps of the calculating of the degree of suitability and the delivering of the service or maintenance order, or a repetition of the method step of the delivering of the service or maintenance order is performed. In the first case, the list of the persons, who have a suitable degree of suitability, is updated. In the second case, the persons, who have a suitable degree of suitability, are simply remembered.

A preferred embodiment of the method of the invention provides that the registering of information for the objects brought along by the person and/or safety equipment worn by the person includes an identification of the objects, or the safety equipment, wherein the identification includes a read-out of identification data from the objects by means of a radio connection, especially RFID, an optical identification of the objects, especially by image recognition by means of a camera, or read-out of a code on the objects, especially a QR-code.

In an advantageous embodiment of the method of the invention, it is provided that the safety equipment has sensors and it is checked at regular intervals by means of the sensors whether the safety equipment is still being worn by the person. For example, the sensors are movement sensors or step sensors, which, for example, are integrated in safety shoes. In this way, it can, on the one hand, be checked whether a person is wearing a certain type of safety equipment. Such is then entered as capability information into the service platform. On the other hand, it can be ascertained, whether the person is still wearing the earlier registered safety equipment, whereby the list of capability information associated with the person is regularly updated.

In a preferred embodiment of the method of the invention, it is provided that information concerning health includes at least one of the following vital parameters of the person:
blood pressure of the person;
heart rate of the person;
body temperature of the person; and
breathing rate of the person.

An advantageous embodiment of the method of the invention provides that information concerning health is registered by a fitness armband worn by the person and/or by a mobile end device brought along by the person. After registration, the information is delivered to the service platform. For this, the fitness armband, or the mobile end device, in given cases, both, are connected with the service platform via the Internet.

In a preferred embodiment of the method of the invention, it is provided that qualification certifications acquired by the persons are supplementally stored in the service platform, and the qualification certifications enter into the calculating of the degree of suitability. Such qualification certifications are obtained by persons, for example, by their attending special schools and concern particular service or maintenance measures. The degree of suitability is increased by a qualification certificate when such is relevant for the particular case of service or maintenance.

A preferred embodiment of the method of the invention provides that a case of service or maintenance of a measuring location relates to the plant component, the field device and/or the communication means of the measuring location.

In an advantageous embodiment of the method of the invention, it is provided that a case of maintenance of the field device is determined in the form of a diagnostic message delivered from the field device, wherein the diagnostic message from the field device is delivered indirectly via a control station to the service platform, or wherein the diagnostic message of the field device is delivered directly, especially via an Internet connection, from the field device to the service platform.

In an advantageous embodiment of the method of the invention, it is provided that upon occurrence of a case of service or maintenance of the measuring location a plurality of service or maintenance orders are created, for which separate degrees of suitability are ascertained and which are delivered to different persons, wherein the individual service or maintenance orders have relative to one another different required objects, health requirements and/or requirements regarding safety equipment required for caring for the case of service or maintenance. For example, a maintenance of a plant component is to be performed, which plant component is located at a great height in the plant. For performing the maintenance, two suitable persons are required. The first person, for example, performs the actual maintenance and has the requisite tools and/or servicing instruments. The second person serves for keeping the first person safe and must bring along safety equipment for this, such as, for example, a rope.

The invention will now be explained in greater detail based on the appended drawing, the sole FIGURE of which shows as follows:

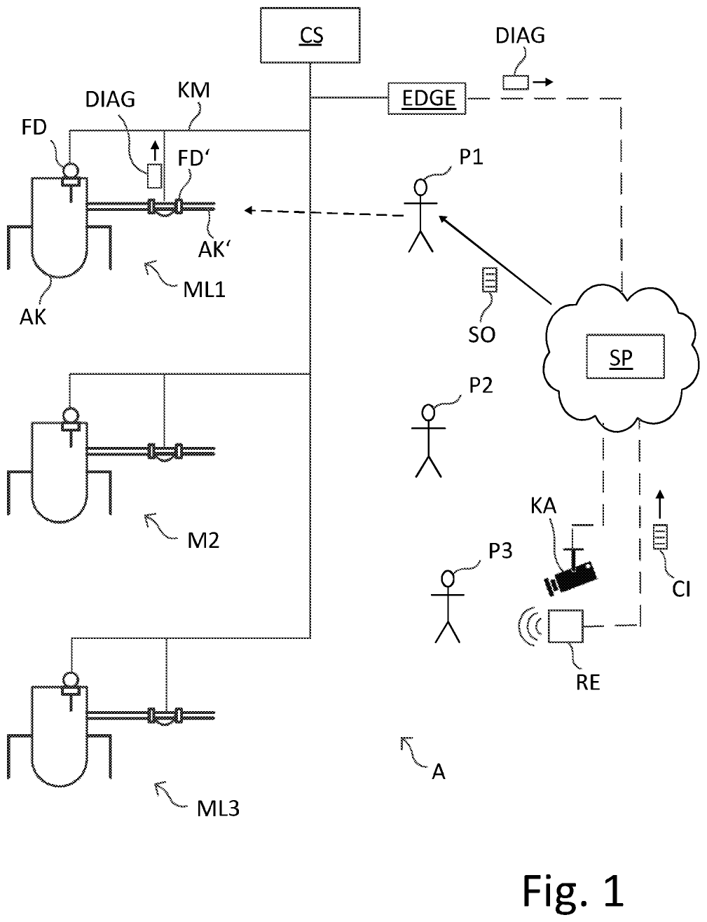

FIG. 1 an example of an embodiment of the method of the invention.

FIG. 1 shows an example of an embodiment of the method of the invention. Shown in the drawing are parts of an automated plant A. In particular, the plant has three measuring locations ML1, ML2, ML3. These involve, in each case, a tank AK and a pipeline AK leading away from the tank. For measuring fill level of the tank, in each case, a field device FD, for example, a fill level measuring device operating by means of radar, is mounted on the tank. Provided, in each case, for measuring flow velocity in the pipeline is a field device FD′, for example, a flowmeter operating according to the Coriolis principle. Each of the field devices FD, FD′ of the measuring locations ML1, ML2, ML3 is placed by means of a communication means KM, for example, a fieldbus of automation technology, in communicative connection with the control station CS of the plant A, in order to transfer the measured process variables of their measuring locations ML1, ML2, ML3 to the control station CS.

The method of the invention is illustrated based on a malfunction of field device FD′:

Field device FD′ detects a defect of one of its mechanical components, which is responsible for the oscillation excitation and creates a high priority diagnostic message DIAG. This case of maintenance is reported in the form of the diagnostic message DIAG to the control station CS of the plant A. The control station CS forwards the diagnostic message DIAG automatically to a service platform SP, which is located in a cloud. Alternatively, the field device FD′ can be embodied, especially, when it is a modern field device ED′ with an IoT interface, to transfer the diagnostic message DIAG on its own via the Internet to the service platform SP. In an additional alternative, the communication means KM is connected with an edge device EDGE. Such is in communicative connection with the service platform SP and forwards the diagnostic message DIAG to the service platform.

The service platform SP registers the diagnostic message DIAG, which has arrived, and compares it with a history located in the service platform SP. The history includes past diagnostic messages from field devices, especially from field devices throughout the plant, and includes objects and, in given cases, safety equipment, which a service technician requires, in order to be able to care for the case of maintenance. Alternatively, the objects and, in given cases, the required safety equipment are defined manually in the control station CS upon arrival of the diagnostic message DIAG and transferred together with the diagnostic message DIAG to the service platform SP. The service platform then creates a service or maintenance order SO, which comprises at least the diagnostic message DIAG, the affected field device ED′ and the needed objects and safety equipment.

The service platform SP contains a list of all persons P1, P2, P3 currently located in the plant A. The list includes capability information CI of all persons P1, P2, P3 in the list. The capability information comprises information concerning all objects, which the particular person P1, P2, P3 brings along, as well as concerning all components of safety equipment, which the particular person P1, P2, P3 is wearing. The objects and the safety equipment of a person P1, P2, P3 are especially registered upon entry of a person P1, P2, P3 into the plant A and noted in the service platform SP. The registering occurs, for example, by means of a video camera KA, which detects the objects/safety equipment optically and/or by means of an RFID-reader RE.

The service platform SP compares the objects and components of safety equipment required for caring for the case of maintenance with the capability information CI stored in the list and calculates a degree of suitability for each of the persons P1, P2, P3. The degree of suitability represents a measure of agreement of the capability information CI of the persons P1, P2, P3 stored in the database with the required objects and the requirements as regards safety equipment contained in the service or maintenance plan SO.

If the degree of suitability for a person P1, P2, P3 is greater than a predetermined value of a degree of suitability, then this person P1, P2, P3 is, in principle, suitable to perform the maintenance. In this example, this selects persons P1 and P3.

Then, the current locational information of the persons P1, P3 is retrieved. Person P1 is located within a predetermined distance from the measuring location ML1 and, consequently, receives the service or maintenance order on a mobile end device carried by person P1. Person P1 is now obliged to go to the measuring location ML1 without delay and to perform the maintenance. After finishing the maintenance, the person P1 certifies the performed maintenance, whereby such is marked “finished” in the service platform.

Besides the capability information CI, “objects” and “protective equipment”, also the health of the person P1, P2, P3 can play a role in the evaluation of the degree of suitability. Especially, when measuring locations ML1, ML2, ML3 are difficult to reach, this information concerning health of the person P1, P2, P3 plays a role in the calculating of the degree of suitability.

The information concerning health comprises at least one of the following vital parameters of the person P1, P2, P3:
blood pressure of the person P1, P2, P3;
heart rate of the person P1, P2, P3;
body temperature of the person P1, P2, P3; and
breathing rate of the person P1, P2, P3.

It can be provided that information concerning health is registered by a fitness armband worn by the person P1, P2, P3 and/or by a mobile end device carried by the person P1, P2, P3. Upon registration, the information is transferred to the service platform. For this, the fitness armband, or the mobile end device, in given cases, both, are connected with the service platform SP via the Internet.

### LIST OF REFERENCE CHARACTERS

A plant
AK, AK′ plant components
CI capability information
DIAG diagnostic message
EDGE edge device
FD, FD′ field devices
KA camera
KM communication means
CS control station
ML1, ML2, ML3 measuring locations
P1, P2, P3 persons
RE RFID-reader
SO service or maintenance order
SP service platform

1. Method for maintaining a measuring location (ML1, ML2, ML3) in an automated plant (A), wherein there exist at the measuring location (ML1, ML2, ML3) at least one plant component (AK, AK′), especially a container and/or a pipeline, in which a process medium is located at least at times, at least one field device (FD, FD′), which is embodied for registering and/or influencing process variables of the process medium, and at least one communication means (KM), which is embodied for transferring to a control station (CS) of the plant (A) the process variables registered by the field device (FD, FD′) and/or diagnostic information produced by the field device (FD, FD′), comprising:
registering capability information (CI) of a person (P1, P2, P3) located in the plant (A) or entering into the plant (A), wherein the capability information (CI) includes information concerning objects brought along by the person (P1, P2, P3), health of the person (P1, P2, P3) and/or safety equipment worn by the person (P1, P2, P3);
sending to and storing the registered capability information (CI) of the person (P1, P2, P3) in a service platform (SP), especially a cloud based service platform;
noting a case of service or maintenance of the measuring location (ML1, ML2, ML3);
creating a service or maintenance order (SO), which includes objects required for caring for the case of service or maintenance, health requirements and/or requirements regarding safety equipment;
calculating a degree of suitability of persons (P1, P2, P3) stored in the service platform (SP) upon noting a case of service or maintenance for the measuring location (ML1, ML2, ML3), wherein the degree of suitability provides a measure of agreement of capability information (CI) for persons (P1, P2, P3) stored in the database with the required objects, the requirements for health and/or the requirements as regards safety equipment contained in the service or maintenance plan; and
delivering the service or maintenance order (SO) to at least one of the stored persons (P1, P2, P3) in the service platform (SP), who has for the noted case of service or diagnosis a degree of suitability, which is equal to or greater than a predetermined degree of suitability.

2. Method as claimed in claim 1, wherein after the noting of the case of service or maintenance, the current locations of those persons (P1, P2, P3), who have a predetermined degree of suitability for the noted case of service or diagnosis, are registered and wherein the service or maintenance order (SO) is delivered only to those persons (P1, P2, P3), who are located within a predetermined distance from the measuring location (ML1, ML2, ML3).

3. Method as claimed in claim 2, wherein the locations are registered by means of a service unit or mobile end device carried by the person (P1, P2, P3).

4. Method as claimed in at least one of the preceding claims, wherein, upon successful removal of the service instance, a certification thereof is delivered to the database by the person (P1, P2, P3), who did the work.

5. Method as claimed in claim 4, wherein, in the case of no certification after a defined time interval after delivery of the service or maintenance order (SO), a repetition of the method steps of the calculating of the degree of suitability and the delivering of the service or maintenance order (SO), or a repetition of the method step of the delivering of the service or maintenance order (SO) is performed.

6. Method as claimed in at least one of the preceding claims, wherein the registering of information for the objects brought along by the person (P1, P2, P3) and/or safety equipment worn by the person (P1, P2, P3) includes an identification of the objects, or the safety equipment, wherein the identification includes a read-out of identification data from the objects by means of a radio connection, especially RFID, an optical identification of the objects, especially by image recognition by means of a camera (KA), or read-out of a code on the objects, especially a QR-code.

7. Method as claimed in at least one of the preceding claims, wherein the safety equipment has sensors and wherein it is checked at regular intervals by means of the sensors whether the safety equipment is still being worn by the person (P1, P2, P3).

8. Method as claimed in at least one of the preceding claims, wherein information concerning health includes at least one of the following vital parameters of the person (P1, P2, P3):
blood pressure the person (P1, P2, P3);
heart rate of the person (P1, P2, P3);
body temperature of the person (P1, P2, P3); and
breathing rate of the person (P1, P2, P3).

9. Method as claimed in at least one of the preceding claims, wherein information concerning health is registered by a fitness armband worn by the person (P1, P2, P3) and/or by a mobile end device brought along by the person (P1, P2, P3) and delivered to the service platform (SP).

10. Method as claimed in at least one of the preceding claims, wherein qualification certifications acquired by the persons (P1, P2, P3) are supplementally stored in the service platform (SP), and the qualification certifications enter into the calculating of the degree of suitability.

11. Method as claimed in at least one of the preceding claims, wherein a case of maintenance of the measuring location (ML1, ML2, ML3) relates to plant components (AK, AK′), the field device (FD, FD′) and/or the communication means (KM) of the measuring location (ML1, ML2, ML3).

12. Method as claimed in at least one of the preceding claims, wherein a case of service or maintenance of the field device (FD, FD′) is determined in the form of a diagnostic message (DIAG) delivered from the field device (FD, FD′), wherein the diagnostic message (DIAG) from the field device (FD, FD′) is delivered to the service platform (SP) via an edge device (EDGE), wherein the diagnostic message (DIAG) from the field device (FD, ED′) is delivered indirectly via a control station (CS) to the service platform (SP), or wherein the diagnostic message (DIAG) of the field device (FD, FD′) is delivered directly, especially via an Internet connection, from the field device (FD, FD′) to the service platform (SP).

13. Method as claimed in at least one of the preceding claims, wherein, upon occurrence of a case of service or maintenance of the measuring location (ML1, ML2, ML3), a plurality of service or maintenance orders (SO) are created, for which separate degrees of suitability are ascertained and which are delivered to different persons (P1, P2, P3), wherein the individual service or maintenance orders (SO) have relative to one another different required objects, health requirements and/or requirements regarding safety equipment required for caring for the case of service or maintenance.
